(19) United States
(12) Patent Application Publication
(10) Pub. No.: US 2007/0269531 A1
(43) Pub. Date: Nov. 22, 2007

(54) Non-Steroid Anti-Inflammatory Agents with Vitamins, Minerals, and Dietary Supplements for the Prevention and Treatment of Primary, Secondary and Tertiary Cancer

(75) Inventors: M. Michael Wolfe, Newton, MA (US); Richard Cotter, Mendham, NJ (US); Alexandra Kropotova, Basking Ridge, NJ (US)

Correspondence Address: WYETH;PATENT LAW GROUP, 5 GIRALDA FARMS, MADISON, NJ, 07940, US

(73) Assignee: Wyeth Patent Law Department, Madison, NJ (US)

(21) Appl. No.: 11/596,360

(22) PCT Filed: May 13, 2005
(86) PCT No.: PCT/US05/16639

§ 371 Date: Jun. 21, 2007

Related U.S. Application Data
(60) Provisional application No. 60/571,597, filed on May 14, 2004.

(51) Int. Cl. A61K 38/16; A61K 36/906

(52) U.S. Cl. 424/649; 424/682; 424/725; 424/775; 514/167; 514/249; 514/274; 514/283; 514/339; 514/34; 514/348; 514/413; 514/452; 514/52; 424/729; 424/757; 424/756; 424/778; 424/764; 424/768; 514/410; 514/8

(57) ABSTRACT

A composition and method for prevention or treatment of cancer The composition comprises a non-steroid anti-inflammatory agent and at least one of nutritional supplement and/or chemotherapeutic agent.

Non-selective and selective non-steroid anti-inflammatory drugs (NSAIDs) have been suggested for use in the prevention and treatment of cancer, and particularly for the prevention and treatment of colorectal cancer. However, NSAID dosages to achieve an amount effective for cancer prevention are often at levels above the dosages normally used when NSAIDs are administered as an analgesic or anti-inflammatory. Taken regularly in these high amounts, cancer prevention benefits may be outweighed by undesirable medical effects caused by large NSAID dosages. Of particular concern for non-selective NSAIDs are effects within the gastro-intestinal tract, including bleeding ulcers and other afflictions that pose a potentially serious health hazard. Other side effects for NSAIDs may include kidney problems, liver toxicity and reduced healing rate.

Except for celecoxib, approved for relieving a tumor burden in patients with Familial Adenomatous Polyposis, NSAIDs have not previously been approved for the treatment of cancer, either alone or in combination with conventional chemotherapeutic agents. It has been discovered that NSAIDs in an amount up to about 1350 mg/day has been shown to be an effective colorectal cancer treatment in animals. In certain cases where cancer patients are already gravely ill, the risk of future gastro-intestinal effects brought on by high doses of NSAIDs are likely to be outweighed by the benefits of current treatment.

To ameliorate the potential negative side-effects of NSAIDs in both cancer treatment and prevention, NSAID dosage may be reduced without compromising efficacy by supplementing the reduced NSAID dosage with various minerals, vitamins, and/or dietary supplements that have been shown to have anti-neoplastic effects. In cases when a need for a high efficacy outweighs safety concerns, such as in populations with a high risk for developing cancer, the combination of NSAIDs in standard doses with minerals, vitamins, and/or dietary supplements is expected to provide a higher efficacy than any single component.

The combination of these components in a dosing regimen is expected to have at least an additive effect. The pharmacokinetic and pharmacodynamic profiles of components having anti-neoplastic properties also suggest the possibility of a synergistic effect that may be even more effective in cancer treatment and prevention than if any one component was taken individually.

The NSAIDs for use in the pharmaceutical compositions and methods of use of the present invention may be selected from any of the following categories:

1. The propionic acid derivatives

2. The acetic acid derivatives

3. The fenamic acid derivatives

4. The biphenylcarboxylic acid derivatives

5. The oxicams

6. Cox-2 inhibitors

Accordingly, the term “NSAID” as used herein is intended to mean any non-steroidal anti-inflammatory compound, including the pharmaceutically acceptable non-toxic salts thereof, falling within one of the six structural categories above.

The specific compounds falling within the foregoing definition of NSAIDs are well known to those skilled in the art and reference may be found in various literature reference sources for their chemical structures, pharmacological activities, side effects, etc. See, for example, Physician's Desk Reference, and The Merck Index.

Propionic acid derivatives for use herein may include ibuprofen, naxproxen, flurbiprofen, fenoprofen, ketoprofen, suprofen, fenbufen, and fluprofen. Of the acetic acid derivatives, exemplary compounds may include tolmetin sodium, zomepirac, sulindac and indomethacin. Of the fenamic acid derivatives, exemplary compounds may include mefenamic acid and meclofenamate sodium. Exemplary biphenylcarboxylic acid derivatives for use in the present invention may include diflunisal and flufenisal. Exemplary oxicams may include piroxicarn, sudoxicam and isoxicam. Exemplary Cox-2 inhibitors may include celecoxib, rofecoxib, meloxicam, and nimesulide. Aspirin may also be used. Of the foregoing NSAIDs, in the practice of the embodiments of the present invention, ibuprofen is exemplified, and may be either selective or non-selective.

Chemotherapeutic modalities to treat cancer, more specifically, colorectal cancer (CRC) although associated with significant toxicity, have provided only a minimal benefit in improving survival. However, it has now been found that the there is a synergistic effect in the treatment of cancer from the combination of traditional chemotherapeutic modalities and ibuprofen. As shown in more detail in the Example, ibuprofen used alone and in combination with various chemotherapeutic agents to treat mice implanted with colorectal cancer cells showed increased safety and efficacy when compared to chemotherapy alone. In this manner, toxicity to healthy cells is minimized.

The chemotherapeutic agents suitable for use in the present invention are cytotoxic drugs that inhibit or disrupt tubulin polymerization, alkylating agents that bind to and disrupt DNA, and agents which inhibit protein synthesis or essential cellular proteins such as protein kinases, enzymes and cyclins. Chemotherapeutic agents commonly used to treat proliferative disorders such as cancer, and which may be used in combination with ibuprofen include, but are not limited to adriamycin, cisplatin, carboplatin, vinblastine, vincristine, bleomycin, methotrexate, doxorubicin, flurouracils, etoposide, taxol and its various analogs, mitomycin, thalidomide and its various analogs, and irinotecan. Preferred cytotoxic drugs are 5 fluoruracil and irinotecan.

Ibuprofen has also been shown to inhibit proliferation and tube formation in human vascular endothelial cells and also decreased angiogenesis in tumors, while inducing tumor cell apoptosis.

In some embodiments benefits may be derived by a treatment regime including one or more NSAID a chemotherapeutic agent and/or a nutritional supplement. Preferred vitamins and minerals that may be used to reduce NSAID dosage for both cancer prevention and cancer treatment include calcium, vitamin D, and folates. Pyridoxines and cobalamines, such as vitamins B6 and B12 respectively, also have similar anti-neoplastic properties that may result in their usefulness in combination with ibuprofen and other NSAIDs. Dietary supplements may include extracts of soy, turmeric, saw palmetto, passion flower, flaxseed, nettle, licorice, ginger, and other similar compounds.

Compositions of the invention may be administered separately as individual components, but may be more preferably formulated and administered in a single dosage form. The single dosage form may be solid (such as tablets, capsules, sachets, trochets and the like), liquid (such as solutions or suspensions) or inhalation aerosols or patches. While solid compounds will typically be administered orally, liquids may be administered orally or by injection, including intravenously. Other dosage forms, such as suppositories, may also be useful. Preferred forms of administration may depend on whether the compositions are administered for cancer prevention or cancer treatment. For example, although intravenous administration may be desirable for a cancer patient undergoing treatment, it would not be desirable as a prevention form for use by the general public, where solid or patch form may be preferable.

Exemplary compositions of the solid dosage forms include bulk powders, tablets, caplets, pellets, capsules, sachets, granules, and any other dosage form suitable for oral administration. The term “tablet” refers equally to a tablet, a caplet or any other solid dosage form which is suitable for oral administration. As those of skill in the art will appreciate, tablets may typically include binders, lubricants, disintegrants, glidants, adsorbents, and other non-active ingredients, in addition to the active components.

Binders are agents used to impart cohesive qualities to the powdered material. Binders impart cohesiveness to the tablet formulation which insures the tablet remaining intact after compression, as well as improving the free-flowing qualities by the formulation of granules of desired hardness and size. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses, and Veegum, and synthetic polymers such as polymethacrylates and polyvinylpyrrolidone.

Lubricants have a number of functions in tablet manufacture. They prevent adhesion of the tablet material to the surface of the dies and punches, reduce inter-particle friction, facilitate the ejection of the tablets from the die cavity and may improve the rate of flow of the tablet granulation. Examples of suitable lubricants include, but are not limited to, magnesium stearate, calcium stearate, stearic acid, glyceryl behenate, talc, sodium lauryl sulfate, sodium stearyl fumarate, polyethylene glycol or mixtures thereof. Generally, the lubricant is present in an amount from about 0.25% to about 5% of the weight of the final composition and more specifically from about 0.5 to about 1.5% of the weight of the final composition.

A disintegrant is a substance, or a mixture of substances, added to a tablet to facilitate its breakup or disintegration after administration. Materials serving as disintegrants have been classified chemically as starches, clay, celluloses, agars, gums and cross-linked polymers. Examples of suitable disintegrants include, but are not limited to, crosscarmelose sodium, sodium starch glycolate, starch, magnesium aluminum silicate, colloidal silicon dioxide, methylcellulose, agar, bentonite, alginic acid, guar gum, citrus pulp, carboxymethyl cellulose, microcrystalline cellulose, or mixtures thereof. Generally, the disintegrant is present in an amount from about 0.5% to about 25% of the weight of the final composition and more specifically from about 1% to about 15% of the weight of the final composition.

Glidants are substances which improve the flow characteristics of a powder mixture. Examples of glidants include, but are not limited to colloidal silicon dioxide, talc or mixtures thereof. Generally, the glidant is present in an amount of from about 0.1% to about 10% of the weight of the final composition and more specifically from 5 about 0.1% to about 5% of the weight of the final composition,

The adsorbent may be, for example colloidal silicon dioxide, microcrystalline cellulose, calcitun silicate or mixtures thereof. Generally, the adsorbent is present in an amount from about 0.05% to about 42% of the weight of the final composition and more specifically from about 0.05% to about 37% of the weight of the final composition.

If desired, other ingredients, such as diluients, stabilizers and anti-adherenlts, conventionally used for pharmaceutical formulations may be included in the present formulations. Other optional ingredients include coloring and flavoring agents which are well known in the art.

The pharmaceutical composition described in the present invention may be formulated to release the active ingredients in a sustained release manner. Various formulations, including elixers, suspensions, tablets, caplets, capsules, and the like are contemplated for dosage forms of these components.

With respect to the dosage amount of the active components in the compositions of the invention, the specific dose will vary depending upon the age and weight of the patient, the severity of the symptoms, the incidence of side effects and the like. For humans, typical effective amounts of NSAIDs, particularly ibuprofen, are expected to be about 1-1200 mg, preferably about 200-800 mg, and most preferably about 100-400 mg.

For calcium, typical effective amounts are expected to be about 1-2400 mg, preferably about 600-1200 mg. For Vitamin D, typical effective amounts are expected to be about 1-4000 IU, preferably about 400-2000 IU, and most preferably about 800-1000 IU. For folate, typical effective amounts are expected to be about 400-1000 mcg, preferably about 400-800 mcg.

Dosing schedules for the components of the composition include taking one dose of each component three times a day, seven days a week, preferably in a single dose form. Preferably, the dosing schedule may be more limited, such as a single dose per day, taken at a regular interval from once a day up to once a week.

By combining lower-dosage amounts of NSAIDs (when compared to dosage levels previously shown effective for the prevention of cancer) with vitamins, minerals and dietary supplements, an optimal dose, dosing regimen, and advantageous risk/benefit ratios for cancer chemoprevention through broad over-the-counter ClistribuLtion can be achieved. These combination doses, as well as higher dosages of NSAIDs alone or in farther combination witli conventional chemotlierapeutic agents can also be used for the treatment of cancer.

### EXAMPLE 1

Example 1 demonstrates the use of ibuprofen in the treatment of cancer.

Nonsteroidal anti-inflammatory drugs (NSAIDs) have been reported to reduce the risk and mortality of colorectal cancer (CRC) by inhibiting the activity of cyclooxygenase (COX). To examine whether non-selective cyclooxygenase inhibitor ibuprofen (IB) has inhibitory effects on tumor growth and liver metastasis in established CRC, a series of mouse and human CRC cancer models have been utilized. IB significantly inhibited cell proliferation in mouse (MC-26) and human (HT-29) CRC cell lines. In vitro angiogenesis assays also indicated that IB decreased both cell proliferation and tube formation. The administration of chow containing 1360 ppm IB, which achieved an average plasma concentration of IB lower than the peak level achieved in humans at therapeutic doses, inhibited tumor growth by 40-82%. Fewer liver metastases were foumd in the IB group compared to the control group. In combination therapy with the standard antineoplastic agents, 5-fluoraracil (5-FU) or irinotecan (CPT-11), tumor volumes in the groups with IB ±CPT-11 or 5-FU were smaller than in the control group. However, death in some of the mice treated with 5-FU occurred earlier than in the control group. These studies demonstrate that ibuprofen, in part by modulating tumor anigiogenesis, decreases both tumor growth and the metastatic potential in mice. The IB doses used, when adjusted for body mass, were in the range of therapeutic human plasma concentrations. Ftulhermore, IB potentiates the anti-tumor properties of CPT-11 and 5-FU in CRC without increasing GI toxicity. Filially, in this experimental model, IB improves survival of mice treated with CPT-11, but not 5-FU.

Colorectal cancer (CRC) is second only to lung cancer as a leading cause of death from malignancy in the United States (Winawer et al., 1997), and up to 50% of patients present at an incurable stage (Moertel et al., 1990). Nearly 130,000 new cases of CRC were diagnosed in the United States in 1999, resulting in 56,600 associated deaths (Ladabaiun et al., 2001). Approximately 6% of Americans will develop CRC during their lifetime, and 2.6% will die from this disease (Saltz et al., 2000).

Chemotherapeutic modalities to treat refractory CRC, although associated with significant toxicity, have provided only minimal benefit in improving survival. Fluorouracil (5-FU), included in several regimens, is generally regarded as the most effective single agent for this disease and is often used as first-line therapy, despite only partial responses to 5-FU achieved in 15 to 20% of patients (Douillard et al., 2000). The probability of tumor response to 5-FU appears to be somewhat greater for patients with liver metastases when chemotherapy is infused directly into the hepatic artery, but intraaiterial treatment is costly and toxic and does not appear to prolong survival (Ekberg et al., 1986; Lorenz et al., 1998). The concomitant administration of folinic acid (e.g. leucovorin) improves the efficacy of 5-FU in patients with advanced CRC. Leucovorin is the active form of the B complex vitamin, folate. Leucovorin is used as an antidote to drugs that decrease levels of folic acid. Folic acid helps red and white blood cell formulation and the synthesis of hemoglobin. By enhancing the binding of 5-FU to its target enzyme, thymidylate synthase. A three-fold improvement in the partial response rate is noted when leucovorin is combined with 5-FU (Douillard et al., 2000). The effect of this combination on survival is marginal, with median survival typically 10-14 months (Bormer M M, 1992). Irinotecan (CPT-11) is a potent inhibitor of topoisomerase I, a nuclear enzyme involved in the unwinding of DNA during replication (Goldberg et al., 2004). Irinotecan has demonstrated antitumor activity against metastatic CRC when used alone as first-line treatment or as second-line treatment after the failure of 5-FU (Douillard et al., 2001}). Prolonged survival was observed when compared to supportive care or infusion of 5-FU and leucovorin as a second-line therapy (Saltz et al., 2000). The FDA has approved the regimen of irinotecan, 5-FU, and leucovorin for the initial treatment of advanced CRC, and many oncologists have now adopted this regimen as standard care for CRC (Sargent et al., 2001; Van Cutsem et al., 2001 a; Van Cutsem et al., 2001b).

Epidemiological studies have demonstrated a 40-50% reduction in mortality from CRC in individuals taking nonsteroidal anti-inflammatory drugs (NSAIDs), which presumably reduce the risk of CRC at least in part by inhibiting cyclooxygenase (COX), a key enzyme involved in the conversion of arachidonic acid to prostaglandins (Sheng et al., 1997). Two isoforms of COX have been identified. These isoenzymes are regulated differently and exhibit distinct functions. COX-1 is expressed constitutively in many cell types, whereas COX-2 is a primary response gene, whose expression may be induced by trauma, growth factors, tumor promoters, and cytokines. COX-2 was first discovered as an oncogene-responsive COX and increased COX-2 expression has been found in up to 85% of colorectal adenocarcinomas, while is undetectable in normal intestinal mucosa (Taketo, 1998b; Taketo, 1998a). A casual relationship between the activity of COX and CRC has been suggested, with COX emerging as a molecular target for chemopreventtion in CRC (Daimenberg and Zakim, 1999).

We have previously demonstrated that the COX-2 selective NSAID rofecoxib is effective in preventing the metastasis of CRC to the liver in mice, an effect achieved via multiple mechanisms (Yao et al., 2003). In the present study, we sought to determine whether the nonselective and relatively inexpensive NSAID ibuprofen would likewise be effective in decreasing the growth and metastatic potential of CRC and might improve mortality in a mouse model of metastatic CRC. Finally, we examined the cellular and molecular mechanisms by which NSAIDs affect CRC tumor growth.

Materials and Methods

Cell Culture

The transplantable mouse CRC cell line MC-26 (Singh et al., 1986) was obtained from Dr. K. K. Tanabe (Massachusetts General Hospital, Boston, Mass.), and the human colorectal adenocarcinoma cell line HT 29 was obtained from ATCC. MC-26 cells and HT-29 were maintained in Dulbecco's modified Eagle's medium (DMEM; Life Technologies, Inc, Gaithersburg, Md.) and McCoy's 5A medium, respectively, supplemented with 10% fetal calf sertum plus antibiotics at 37° C. in a humidified atmosphere of 95% air/5% C02. Human umbilical vein endothelial cells (HUVECs) was purchased from Canibrex (Walkersville, Md.) and maintained in EGM-2 medium (Cambrex).

Prostaglandin E₂ Assay

Prostaglandin E₂ (PGE₂), the major metabolite of arachidonic acid metabolism, was measured by ELISA (Cayman Chemical, Ami Arbor, Mich.), using conditional cell culture medium, according to the protocol provided by manufacturer. Measurements were made in triplicate in separate experiments.

In Vitro Cell Proliferation Assay (MTT Assay)

In Vitro cell proliferation was assessed using a CellTiter 96®AQ One Solution Cell Proliferation Assay kit (Promega). 1-3×10⁴ cells/ml were plated in 96-well microtiter plates and incubated overnight. Cells were then treated with various concentrations of ibuprofen for 24, 48, or 72 h. 20 μl of MTS tetrazolium solution were added to each well, and the cells were further incubated at 37° C. for 1 h, at which time the reactions were stopped by the addition of 10% SDS. The absorbance at wavelength 490 nm was measured using an EX800 microplate reader (Bio-Tek, Highland, Wis.).

In Vitro Angiogenesis Assay

An in vitro anigiogenesis assay was performed according to the protocol provided by manufacturer (Chemicon, Temecula, Calif.) and described previously (Strasly et al., 2004; Yanailandra et al., 2004). In brief, HUVECs were incubated in endothelial cell growth media overnight. 2 ml of cell suspension (1-2×10⁵ cell/ml) were then harvested and loaded on pre-coated Matrigel basement membrane matrix dishes and cultured in EGM-2 medium containing ibuprofen (1000 μM at 37° C. for 0.5, 2, 6, or 24 hours, followed by staining with hematoxylin for 20 min. Microscopic digital images were taken, and cumulative tube length was measured using Spot™3.5.6.

Immunohistochemistry

To assess anigiogenesis in tumor tissues, frozen sections were made and microvessels were stained with anti-mouse CD31 mnonocolonal antibodies (BD transduction laboratories). The specimens were incubated with secondary antibody for 1 h at 37° C. and stained by the avidin-biotin peroxide complex (ABC) method using an ABC staining kit (Santa Cruz Biotech, Santa Cruz, Calif.). They were visualized by 3,3′-diammobenzidme (DAB) and counlterstained with hematoxyhn.

Western Blot Hybridization

Mouse COX-1, COX-2 monoclonal antibodies were purchased from BD transduction laboratories (Lexington, Ky.). To extract protein, MC 26 cells were harvested and lysed in RIPA buffer (PBS, 1% NP-40, 0.5% sodium deoxycholate, 0.1% SDS, 100 ng/ml PMSF, 66 ng/ml aprotiniin). To extract protein from tissues, 0.1 g of tumor tissue was put in 2.0 ml of cold protein extract (RIPA) buffer and homogenized for 1 min with a Polytron-Aggregate (Kinmatica, Luzern, Switzerland). After removal of cellular debris by centrifugation, total protein extracted from cells or tissues was determined by BCA protein assay (Pierce chemical, Rockford, Ill.). Protein was mixed with gel loading buffer (50 mM Tris pH 6.8, 2% SDS, 10% glycerol, 2% 2-mercaptoethanol, 0.1% bromphenol blue) and heated for 10 min at 100° C. Samples containing 5-20 μg protein were loaded onto 10-12% SDS-PAGE gel and then electrophoretically transferred to polyvinylidene difluoride membrane in transfer buffer (25 mM Tris, 190 mM glycine, 20% methanol). The blots were blocked with 7% dry milk for 1 h at room temperature and incubated with the first antibody overniglht. The blots were then washed 3 times for 15 min each in Tris-buffered saline containing 0.05% Tween-20. The blots were further incubated with the anti-mouse IgG antibody (Sigma Chemical Co., St. Louis, Mo.) for 1 h at room temperature. After washing 3 times, blots were incubated with luminous ECL reagent (Pierce chemical, Rockford, Ill.) for 10-20 seconds and exposed to Kodak X-ray film. Protein bands were identified by protein size and positive control provided by BD transduction laboratories (Lexington, Ky.). The amount of protein expression was quantified by Bio-Rad GS 700 Imaging Densitometer.

Blood Sampling and Serum Ibuprofen Concentration

Six-week old male BALB/C mice were fed with a formulation containing 85 mg ibuprofen per kg chow, 170 mg/kg, 340 mg/kg, and 1360 mg/kg. Ibuprofen was purchased from Sigma, and pellet-formed chow containing ibuprofen was prepared by Harlan Teklad (Madison, Wis.). Blood was drawn on day 7 and 14 by tail bleeding, and serum was separated from the whole blood and frozen at −70° C. until analysis.

Serum concentrations of ibuprofen were analyzed by MDS Pharma Services Inc., Saint-Laurent, Quebec, Canada. The plasma samples were analyzed by a quantified liquid chromatography-tandem mass spectrometry (LC/MS/MS) method. Sample preparation was performed using protein precipitation.

The precursor-to-production transitions used were as follows: Ibuprofen at m/z 205.2→161.1 and internal standard (Phenacetin I.S.) at m/z 178.2→148.72. The retention times (t_{R}) for ibuprofen and internal standard were 1.82 and 1.95 min, respectively. A 1511-μL aliquot of internal standard working solution (3000 ng/mL of Phenacetin I.S. in acetonitrile) was added to 25 μL of plasma. Each sample was then vortexed and centrifuged for 15 min at a speed of 13,000 rpm at 4° C.

After centrifugation, 150 μL of the supernatant was transferred into an HPLC vial, and aliquots of 5 μL were injected using a Perkin Elmer 200 Series autosampler (Perkin Elmer: Norwalk Conn.). The loading and analytical columns were a Zorbax Extend C18 (4.6×12.5 mm, 5 μdp; Agilent Technologies: Palo Alto, Calif.) and a Waters Xterra C 18 (3.9×20 mm, 3.5 μdp; Waters Corporation: Milfaird, Mass.), respectively. The loading mobile phase (with gradient loading) consisted of 100/0 (v/v) 10 mM ammonium acetate/acetonitrile that was held for 1.00 min. The acetonitrile was increased to 90% at 1.01 min and held for 1.00 min. Original conditions were resumed at 2.40 min. Initial conditions for gradient elution were carried out with a mobile phase of 100% (v/v) 0.1% arnmmonium solution in water/acetonitrile where was held for 1.00 min. The acetonitrile was increased to 70% at 1.50 min and held for 0.50 min., and at 2.10 min, the acetonitrile was increased to 90% and held for 1.00 min. Original conditions were resumed at 3.10 min. A flow rate of 1.0 mL/min was used throughout the run, with switch events occurring at 1.00 min (loading) and 2.40 min (injecting) for a total run-time of 4.00 min. The column effluent was analyzed by selected reaction monitoring using a triple quadruple mass spectrometer (Micromass Quattro Ultima; Manchester, UK) equipped with an electrospray ion source operating in negative ion mode.

Animal Models and Experimental Designs

Six-week old male BALB/C mice and BALB/C nude mice (T-cell deficiency) were obtained from Taconic (Germantown, N.Y.). We utilized our previously described mouse colon cancer model was utilized, as described (Yao et at., 2002; Yao et al., 2003; Yao et al., 2004). Briefly, MC-26 model cells were harvested from subconfluent cultures by exposure to trypsin-EDTA (Life Technologies, Inc, Gaithersburg, Md.) for 3 min, centrifugation at 300 g for 15 min at room temperature, and then resuspension in serum-free DMEM or Halik's Balanced Salt Solution (Life Technologies, Inc, Gaithersburg, Md.) to yield a final concentration of 5×10⁶ cells/ml. For the subcutaneous MC-26 model, using a 27-gauge needle and a 1-ml syringe, 100 μl of MC-26 tumor cells suspension were injected subcutaneously in the flank of BALB/c mice. For the HT-29 human xenograft CRC model, tumor slices of approximately 1.0 mm in diameter, harvested immediately from a xenograft HT-29 tumor, were implanted in the flanks of the animals. All animal studies were conducted using a protocol approved by the Institutional Animal Care and Use Committee of Boston University Medical Center.

To determine the inhibitory effect of ibuprofen, as a single agent, on tumor growth, immediately after cells were implanted sub cutaneously into mice on day 1, we randomly divided the mice into two groups and fed them with chow containing either no ibuprofen (control) or ibuprofen at a concentration of 1360 mg/kg (10 mice/group). In both MC-26 and HT-29 tumor models, tumor size was determined by measuring the longest and shortest diameters of the tumor. Tumor volume (mm³) was calculated using the standard formula: tumor volume=(shortest diameter)²×(longest diameter)×0.5.

To examine the effects of ibuprofen on experimental colorectal cancer liver metastasis, 2×10⁴ cells/ml MC-26 cells were injected into the subsplenic capsule of six-week-old BALB/c. Mice were randomly divided into a control group and an ibuprofen group (1360 mg/kg ibuprofen) on day 0 and sacrificed on day 14. The incidence and extent of liver metastasis were then recorded.

To determine possible synergistic effects of ibuprofen with clinically used standard chemotherapy, mice bearing subcutaneous MC-26 tumors were administered 1360 mg/kg ibuprofen (dose at which subcutaneous tumor growth was significantly inhibited) and the chemotherapeutic regents 5-fluoruracil (5-FU, Sigma) plus leucovorin (LV, Sigma) rescue and irinotecan (CPT-11, Pharmacia/Upjohn) by intraperitoneal injection (IP). Starting on day 7 after tumor cell inoculation, mice were randomly divided into eight groups as follow:

Group 1: control; control chow; 0.9% NaCl injected intraperitoneally (IP) on days 9, 11, 15, 18, 23, and 28; and 5% IP glucose on days 8, 12, 15, 19, 22, and 25.

Group 2: ibuprofen alone; ibuprofen (1360 mg/kg) chow; 0.9% NaCl IP on days 9, 11, 15, 18, 23, and 28; and 5% glucose IP on days 8, 12, 15, 19, 22, and 25.

Group 3: 5-FU/LV alone; control chow; 5-FU 30 mg/kg and leucovorin (LV) 50 IP mg/kg on days 9, 11, 15, 18, 23 and 28; and 5% glucose IP injection on days 8, 12, 15, 19, 22, and 25.

Group 4: irinotecan alone; control chow; irinotecan 30 mg/kg on days 8, 12, 15, 19, 22, and 25; and 0.9% NaCl on days 9, 11, 15, 18, 23, and 28.

Group 5: ibuprofen/5-FU/LV; ibuprofen (1360 mg/kg) chow; 5-FU 30 mg/leg and leucovorin 50 mg/kg on days 9, 11, 15, 18, 23, and 28; and 5% glucose on days 8, 12, 15, 19, 22, and 25.

Group 6. ibuprofen/irinotecan; ibuprofen (1360 mg/kg) chow; irinotecan 30 mg/kg on days 8, 12, 15, 19, 22, and 25; and 0.9% NaCl on days 9, 11, 15, 18, 23, and 28.

Group 7: 5-FU/LV/irinotecan; control chow; 5-FU 30 mg/kg and leucovorin 50 mg/kg on days 9, 11, 15, 18, 23 and 28; and irinotecan 30 mg/kg on days 8, 12, 15, 19, 22, and 25.

Group 8: ibuprofen/5-FU/LV/irinotecan; ibuprofen (1360 mg/kg) chow; 5-FU 30 mg/kg and leucovorin 50 mg/kg on days 9, 11, 15, 18, 23 and 28; and irinotecan 30 mg/kg on day 8, 12, 15, 19, 22, and 25.

Mice were closely monitored, and studies were terminated when all mice in control group had died. The causes of death included spontaneous death due to tumor burden or drug toxicity or sacrifice by the investigators within 24 h of the determination that a mouse was demonstrating ill behavior. The date and number of animal in each group were recorded for survival analysis.

Statistical Analysis

One-way ANOVA was performed for comparing tumor volume and weight and followed by Tukey's procedure for pair wise comparison. The Student t test was performed to analyze MTT values, densitometric values of Western blot bands, CD31 microvessel staining and tube length of in-vitro angiogenesis assay among different conditions compared with data obtained under control conditions. Survival analysis was performed using the Kaplan-Meier method and log-rank tests. Statistical significance was assigned if P<0.05.

Results

Cell Proliferation, COX Expression and Prostaglandin E2 Production

COX-1 and COX-2 protein expression was determined by Western hybridization analysis. After normalizing data using cytoskeletal actin, no significant differences in COX-1 and COX-2 concentration among the cells treated with various concentrations of ibuprofen at 24 h were detected. In contrast, the production of PGE₂: the major product of the arachidonic acid pathway, was significantly reduced in cells treated with various concentrations of ibuprofen. After 24 h of incubation with ibuprofen, maximum inhibition of MC-26 cell PGE₂ levels was detected when cells were incubated in an ibuprofen concentration of 1000 μM, at which time PGE₂ levels were diminished by 70.1% compared to control (80.0 pg/mL vs. 268.5 pg/mL; P<0.01). A similar pattern was seen when HT-29 cells were cultured in medium containing ibuprofen. After 24 h, PGE₂ levels were diminished by 81.1% compared to control (18.0 pg/muL vs. 95.6 pg/mL.

The addition of ibuprofen to MC-26 and HIT-29 cells led to a concentration-dependent reduction in cell proliferation, as assessed by MTT assay. Ibuprofen at concentrations of 125 μM, 250 μM, 500 μM, and 1000 μM decreased MC-26 cell proliferation by 1.3%, 5.8%, 11.3% axd 39.8% (P<0.01) at 72 h, respectively, compared to cells incubated in the absence of ibuprofen. In HT-29 cells, after 72 h treatment maximum inilhibition of cell proliferation was detected using 1000 μM ibuprofen, at which time MTT levels were diminished by 26.7%, compared to control (P<0.01).

In Vitro Anglogenesis

To assess the effect of ibuprofen on the capacity of in vitro neovascularization, HUVEC cell MTT and tube formation assays were performed. After 24 h of treatment, maximum inhibition of cell proliferation was detected using 1000 μM ibuprofen, at which time MTT levels were diminished by approximately 80%, compared to control (P<0.01). Using the tube formation assay, the 6-h tube lengths were 155.4±21.3 μm in control and 94.0±24.9 μm in 1000 μM ibuprofen (40% reduction, P<0.01). The 24-h tube lengths were 76.2±13.5 μm in control and 9.40±6.95 μm in 1000 μM ibuprofen (88% reduction, P<0.01.

Effect of Ibuprofen on Tumor Growth and Liver Metastasis

Rat chow containing different concentrations of ibuprofen (85 mg ibuprofen in 1 kg rat chow, 170 mg/kg, 340 mg/kg, 680 mg/kg, and 1360 mg/kg) was prepared initially to evaluate ibuprofen plasma concentrations in nice. Blood was collected by tail bleeding on days 7. and 14, and ibuprofen plasma concentrations were measured by HPLC. Following the administration of 300 mg of ibuprofen (in capsule form) to fasting humans, plasma ibuprofen concentrations are approximately 27,000 ng/ml at peak concentration (Halsas et al., 1999). We determined that mice fed chow containing 1360 mg/kg ibuprofen achieved a plasma ibuprofen concentration of approximately between 18,760±7,270 ng/ml and 20,978±5,966 ng/ml, which is within the level obtained after administration of 300 mg of the drug to humans. Gross necroscopic examination revealed no evidence of gastroduodenal mucosal injury in nice who consumed the chow containing ibuprofen. We therefore utilized chow containing 1360 mg/kg of ibuprofen during the in vivo studies assessing tumor growth and metastasis.

MC-26 cells were injected subcutaneously in BALB/C nice on day 0. On day 1, mice were randomly divided into two groups with 10 mice per group: control chow and chow containing ibuprofen 1360 mg/kg. Ibuprofen significantly inhibited tumor growth in mice. The tumor volumes were 39.9±19.6 mm³, 166.0±144.0 mm³, 276.5±227 mm³, and 573.1±376 mm³ on days 10, 14, 17, and 21, respectively, in the control group. Conversely, in the ibuprofen group, the tumor volumes were 21.9±17.3 mm³, 59±57 mm³, 48±57 mm³, and 101±161 mm³ on days 10, 14, 17, and 21, respectively, corresponding to 47.4%, 64.4%, 82.6%, and 82.3% decrease in trunor volume. The average body weight was significantly lower on day 21 in the control group (20.3±2.6 g) than in ibuprofen-treated mice (24.1±1.73 g), corresponding to a 16% body weight reduction in the control group (P<0.01).

To determine whether the above anti-tumor effects of ibuprofen are reproducible in human CRC cancer cells, the HT-29 human CRC xenograft model was established by the subcutaneous implantation of tumor fragments approximately 1.0 mm in diameter into the flanks of nude mice. Treatment started on day 1 and was completed on day 42 (week 7). Tumor volume in the ibuprofen group, on day 42, was reduced by 40% compared to control mice (164.4±99.3 mm³ vs. 410.9±233.2 mm³, P<0.05). However, in contrast to MC-26 cells, no significant difference in body weight was detected between the two groups of mice with implanted HT-29 cell tumors throughout the period of investigation.

The effects of ibuprofen on the metastatic potential of CRC were examined in an experimental liver metastasis model established by the subsplenic capsule injection of MC-26 cells (Yao et al., 2003). Mice were randomly divided into two groups (5 mice/group) and were fed control chow or chow containing ibuprofen 1360 mg/kg for 14 days. Visible metastatic hepatic nodules were more prevalent and larger in the control group than in ibuprofen-fed mice.

Immunohistochemical analysis of frozen tumor sections demonstrated a decrease in the number of CD31 positive microvessels in mice fed chow containing ibuprofen. The number of microvessels was 37.7±15.1/field in the control group (n=8) and 26.4±10.3/field (n=7) in the ibuprofen group, representing approximately a 30% reduction in the latter group of animals (P=0.059).

Effect of Ibuprofen and Other Anti-Neoplastic Agents in the Treatment of CRC

As shown above, ibuprofen possesses inhibitory effects on both tumor growth and liver metastasis. Additional studies were performed to determine whether ibuprofen, in combination with standard chemotherapy, has any synergistic or additive effects on tumor growth. The mice received subcutaneous flank injections of MC-26 cells on day 0, but did not receive any treatment for the first week. From day 7 to day 28, mice received combination therapy, and the study was continued until all control mice had died (day 50). Tumor growth was significantly inhibited in all groups of animals receiving ibuprofen, 5-FU, or CPT-11, either alone or in various combinations, compared with the control group (1491.3±862 mm³). The maximal inhibition was found in the group with a combination of ibuprofen, CPT-11, and 5-FU/leucovroin (110.1±80 mm³), representing a 92.6% reduction in tumor volume (P<0.01). The tumor voltume in this group was significantly smaller than in the groups of mice treated with ibuprofen alone (517±377 mm³ and 5-FU alone (499±234 mm³) (P<0.01).

The effects of the various treatment regimens on body weight were evaluated on day 28. In only three of the animal groups (ibuprofen alone, CPT-11 alone, and the combination of ibuprofen and CPT-11) body weight remained stable (average body weight of 23.1±1.72 g). In addition to significant weight loss in the control group (19.65±1.24 g), body weight decreased in all animals receiving 5-FU/leucovorin. The average body weight in these groups was only 79.8% of groups not receiving 5-FU (18.44±1.88 g vs. 23.1±1.72 g; P<0.05).

The survival of mice receiving ibuprofen alone, CPT-11 alone, or ibuprofen/CPT-11 was significantly prolonged compared to control mice and mice receiving other treatment regimens. The overall survival rates were 80% in mice receiving ibuprofen alone and CPT-11 alone, and 90% in mice treated with ibuprofen/CPT-11. In contrast, much like its effects on body weight, survival appeared to be adversely affected by treatment with 5-FU. Some mice in the groups with 5-FU died earlier than those in the control group, and the overall survival rates at day 50 in mice treated with 5-FU alone, ibuprofen/5FU, 5-FU/CPT-11, and ibuprofen/CPT-11/5-FU were 30%, 20%, 40% and 30%, respectively.

Discussion

A growing body of experimental evidence demonstrates the contribution of COX-2 overexpression in CRC tumorigenesis. Nonselective COX inhibitors, which inhibit both isoforms, have been reported to prevent tumorigenesis. The use of nonselective COX inhibitors is associated with adverse gastrointestinal (GI) events, such as upper GI bleeding, whereas COX-2 selective inhibitors are thought to exert their anti-inflammatory and antineoplastic properties with diminished toxicity (Hirata et al., 1997; Wolfe, 1998; Wolfe et al., 1999; Lichtenstein and Wolfe, 2000). Nevertheless, ibuprofen, a generic and relatively inexpensive nonselective COX inhibitor, is still widely used in clinical practice. Despite its widespread use throughout the world for over 40 years, the potential benefit of this agent in preventing or treating CRC has not been assessed. In the present study, we evaluated the efficacy of ibuprofen in attenuating the growth and liver metastasis of CRC, as well as potentiating the effects of standard CRC chemotherapy.

As mentioned previously, peak plasma ibuprofen concentrations in humans administered a single dose of 300 mg (Halsas et al., 1999) are approximately 27,000 ng/mil or greater. This level is considerably lower than those achieved using ibuprofen doses commonly employed for analgesia or fever-control, which are generally 1.2-2.4 g daily in divided doses. Moreover, the use of low-dose ibuprofen is known to be associated with a very low incidence of serious GI toxicity. A meta-analysis (Lewis et al., 2002) of three retrospective case-control studies on serious upper GI bleeding reported that ibuprofen had the lowest odds ratio and safest side-effect profile among nonselective NSAIDs (OR=1.7, 95% confidence interval 1.1-2.5), followed by diclofenac (4.9; 3.3-7.1), naproxen (9.1; 6.1-13.7), piroxicam (13.1; 7.9-21.8) and ketoprofen (34.9, 12.7-96.5). Most importantly, the risk ratio for ibuprofen at a total dose of 1200 mg/day or lower was only 1.1 (CI 0.6-2.0), comparable to a control population.

The dose of ibuprofen used in the present animal study was even lower than those employed in humans, which likely accounts for the absence of GI mucosal injury. Previous pharmacokinetic data (Corpetti et al., 1998; Konstanl et al., 2003) in healthy humans has shown that long-term treatment with high-dose ibuprofen (14 mg/kg) achieved peak plasma concentrations (Cₘₐₓ) of 65,000 ng/ml. Moderate doses of 7 mg/kg achieved Cₘₐₓ of 34,000 ng/ml, and low doses of 2.6 mg/kg achieved Cₘₐₓ of 15,800 ng/ml. In our study, mice fed a 1360 ppm ibuprofen diet achieved concentrations between 18,760±7,270 ng/ml and 20,978±5,966 ng/ml. The concentrations achieved in our animal study were thus comparable to the lower portion of the therapeutic range in humans.

Ibuprofen at the low dose of 1360 ppm, as a single agent, effectively inhibited both MC-26 and HT-29 tumor growth in vivo, as well as liver metastasis in the MC-26 model. No GI mucosal injury was detected in these mice, and the overall inhibition of tumor growth was similar to the level reported in a previous study examining the effects of selective COX-2 inhibition with rofecoxib (Yao et al., 2003). Our observations are consistent with the notion that the lack of GI toxicity was due to the low ibuprofen concentrations required to achieve an antineoplastic effect, compared to the markedly higher concentrations, associated with greater toxicity, needed to attain analgesic and anitipyretic effects in humans. Additionally, ibuprofen, because it is available without prescription, is considerably less expensive than selective COX-2 inhibitors. Using the on-line pricing site of a major pharmacy (wvw.CVS.com), we found the following prices listed for a one month supply (30 capsules) of these medications: ibuprofen 600 mg ($9.99), in rofecoxib 25 mg ($95.59), and celecoxib 200 mg ($92.99). Thus, the prolonged use of this relative inexpensive drug might confer significant health benefits with a considerable cost saving.

Currently, the regimen of irinotecan, 5-FU, and leucovorin for the initial treatment of advanced CRC has been adopted as standard of care by many oncologists. To test any potential synergistic or additive effects of ibuprofen in combination with these agents on tumor growth and overall survival, we initiated treatment on day 7, a time at which subcutaneous tumor growth approached approximately 0.5 cm in diameter. The treatment duration was three weeks, from day 7 to day 28. Although tumor growth was significantly inhibited in each therapeutic group, each group treated with 5-FU manifested severe toxicity associated with remarkable body weight loss. Survival analysis showed that some of the 5-FU treated mice, even with a low tumor burden, were starting to die by the third week of treatment (week 4 of the study). These mice had an even shorter survival than the ones in the control group. In contrast, the survival of mice receiving ibuprofen alone, CPT-11 alone, or ibuprofen/CPT-11 combination was significantly prolonged compared to control mice and mice receiving other treatment regimens.

Multiple molecular and cellular mechanisms are involved in exerting the antiinflammatory and anti-tumor effects of NSAIDs. These usually divided into two major categories: COX-dependent (Sheng et al., 1997) and independent pathways (Grosch et al., 2001; Kundu et al., 2002; Babbar et al., 2003). It has been long assumed that the antiproliferative effects of NSAIDs are dependent upon inhibition of COX activity and prostaglandin synthesis. COX-2 selective inhibitors are able to induce cell differentiation and cell apoptosis (Tsujii and DuBois, 1995) and inhibit angiogenesis (Tsujii et al, 1998) and cell invasion/migration (Tsujii at al., 1997; Cheng et al., 2002). Using rofecoxib, we recently demonstrated (Yao et al., 2003), by Western hybridization analysis of primary tumor, decreases in proteins expression of COX-2, b-β-catenin, cyclin D1, vascular endothelial growth factor (VEGF), matrix metalloproteinases (MMP)-2 and MWP-9. These observations indicate that multiple mechanisms are involved in exerting the beneficial effects of COX-2 selective inhibition on tumor growth and metastasis. In the present study, we focused on the effects of ibuprofen on tumor angiogenesis and, like selective COX-2 inhibition, demonstrated the marked inhibitory effect of ibuprofen not only on prostaglandin synthesis and tumor/endothelial cell proliferation, but also on anigiogenesis both in vitro and in vivo.

In conclusion, ibuprofen, within or below the range of therapeutic human plasma concentrations, decreases both tumor growth and the potential for liver metastasis, at least in part, by modulating tumor angiogenesis in the CRC mouse model. Ibuprofen potentiates the tumor properties of CPT-11 and 5-FU in CRC without increasing GI mucosal injury, and improves survival of mice treated with CPT-11, but not 5-FU. Ibuprofen may thus prove to be adjunct to standard chemotherapy regimens.

Although the example refers to the prevention and treatment of colorectal cancer, other types of cancers are also expected to respond to treatment with NSAIDs and/or NSAIDs combined with vitamins, minerals and/or dietary supplements and/or combined with one or more chemotherapeutic agent.

The present invention is not to be limited in scope by specific embodiments described herein or in the documents which form the examples of this provisional application. Indeed various modifications of the invention in addition to those described will become apparent to those spilled in the art from the foregoing description and accompanying documents.

1. A method for prevention or treatment of cancer, the method comprising administering to an individual an effective amount of a least one non-steroid anti-inflammatory agent and an effective amount of at least one nutritional supplement selected from the group consisting of calcium, vitamin D, folates, pyridoxines, cobalamines, and combinations thereof.

2. A composition for prevention or treatment of cancer comprising a effective amount of a least one non-steroid anti-inflammatory agent and an effective amount of at least one nutritional supplement selected from the group consisting of calcium, vitamin D, folates, pyridoxines, cobalamines, and combination thereof.

3. The composition of claim 2 further comprising a dietary supplement extract obtained from the group consisting of soy, turmeric, saw palmetto, passion flower, flaxseed, nettle, licorice, ginger and combinations thereof.

4. The composition of claim 2 wherein the composition is formulated as a single dosage unit.

5. A method for prevention or treatment of cancer, the method comprising administering to an individual an effective amount of a least one non-steroid anti- inflammatory agent and an effective amount of at least one chemotherapeutic agent selected from the group consisting of adriamycin, cisplatin, carboplatin, vinblastine, vincristine, bleomycin, methotrexate, doxorubicin, flurouracils, etoposide, taxol, taxol analogs, mitomycin, thalidomide, thalidomide analogs, irinotecan and combinations thereof.

6. A composition for prevention or treatment of cancer comprising a effective amount of a least one non-steroid anti-inflammatory agent and at least one chemotherapeutic agent selected from the group consisting of adriamycin, cisplatin, carboplatin, vinblastine, vincristine, bleomycin, methotrexate, doxorubicin, flurouracils, etoposide, taxol, taxol analogs, mitomycin, thalidomide, thalidomide analogs, irinotecan and combinations thereof.

7. The composition of claim 6 further comprising a nutritional supplement selected from the group consisting of calcium, vitamin D, folates, pyridoxines, cobalamines and combinations thereof and wherein the chemotherapeutic agent is a selected from the group consisting of 5 fluorouracil, irinotecanone and combinations thereof.

8. The composition of claim 7 wherein the composition is formulated as a single dosage unit.
